(19) United States
(12) Patent Application Publication
(10) Pub. No.: US 2006/0241720 A1
(43) Pub. Date: Oct. 26, 2006

(54) Graphical representation of pain therapy

(76) Inventors: Carla M. Woods, Beverly Hills, CA (US); John D.H. King, Los Angeles, CA (US)

Correspondence Address: SIDLEY AUSTIN BROWN & WOOD LLP (LAIP GROUP), 555 W. FIFTH ST., SUITE 4000, LOS ANGELES, CA, 90013, US

(21) Appl. No.: 11/115,644

(22) Filed: Apr. 26, 2005

(51) Int. Cl. A61N 1/34

(52) U.S. Cl. 607/46; 600/557

(57) ABSTRACT

A graphical display for use in a stimulation session includes a human figure depiction and a depiction of the one or more implanted electrodes. The depictions aid in pain mapping and in programming effective electrode configurations to be supplied in a pain stimulation session. Pain maps, paresthesia maps, and/or stimulation sets may be associated and stored in a database.

### BACKGROUND OF THE INVENTION

The present invention relates to tissue stimulation systems and more particularly to display interfaces having graphical depictions used during a stimulation session. One example of a stimulation system is a spinal cord stimulation system (“SCS”). Spinal cord stimulation is a well accepted clinical method for reducing pain in certain populations of patients. An SCS system typically includes an Implantable Pulse Generator (IPG) or a radio-frequency (RF) transmitter and receiver, electrodes, electrode leads, and when necessary, lead extensions. The electrodes are implanted along the dura of the spinal cord, and the IPG or RF transmitter generates electrical pulses that are delivered, through the electrodes, to the dorsal column and dorsal root fibers within the spinal cord. Individual electrode contacts (the “electrodes”) are arranged in a desired pattern and spacing in order to create an electrode array. Individual wires within one or more electrode leads connect with each electrode in the array. The electrode leads exit the spinal column and attach to one or more electrode lead extensions, when necessary. The electrode leads or extensions are typically tunneled around the torso of the patient to a subcutaneous pocket where the IPG or RF-receiver is implanted.

Spinal cord stimulators and other stimulation systems are known in the art. For example, an implantable electronic stimulator is disclosed in U.S. Pat. No. 3,646,940 issued Mar. 7, 1972 for “Implantable Electronic Stimulator Electrode and Method” that provides timed sequenced electrical impulses to a plurality of electrodes. As another example, U.S. Pat. No. 3,724,467 issued Apr. 3, 1973 for “Electrode Implant For The Neuro-Stimulation of the Spinal Cord,” teaches an electrode implant for the neuro-stimulation of the spinal cord. A relatively thin and flexible strip of physiologically inert plastic is provided as a carrier on which a plurality of electrodes are formed. The electrodes are connected by leads to an RF receiver, which is also implanted.

In U.S. Pat. No. 3,822,708, issued Jul. 9, 1974 for “Electrical Spinal Cord Stimulating Device and Method for Management of Pain,” another type of electrical spinal cord stimulation device is taught. The device disclosed in the '708 patent has five aligned electrodes, which are positioned longitudinally on the spinal cord. Electrical pulses applied to the electrodes block perceived intractable pain, while allowing passage of other sensations. A patient operated switch allows the patient to adjust the stimulation parameters.

An SCS system treats chronic pain by providing electrical stimulation pulses through the electrodes of an electrode array located at the distal end of a lead placed epidurally next to a patient's spinal cord. The combination of electrodes used to deliver stimulation pulses to the targeted tissue constitutes an electrode configuration. In other words, an electrode configuration represents the polarity, being positive, negative, or zero, and relative percentage of the current or voltage provided through each of the electrodes.

Electrode arrays used with known SCS systems may employ between 1 and 16 electrodes on a lead. Electrodes are selectively programmed to act as anodes, cathodes, or left off, creating an electrode configuration. The number of electrodes available, combined with the ability to generate a variety of complex stimulation pulses, presents a huge selection of electrode configurations and stimulation parameters (together referred to herein as “stimulation sets”) to the clinician. When an SCS system is implanted, a procedure is performed to select one or more effective stimulation sets for a particular patient. Such a session of applying various stimulation parameters and electrode configurations may be referred to as a “fitting” or “programming” session. Additionally, a series of electrode configurations to be applied to a patient may be organized in a steering table or in another suitable manner.

Other parameters that may be controlled or varied in SCS are the frequency of pulses provided through the electrode array, pulse width, and the strength (amplitude) of pulses delivered. Amplitude may be measured in milliamps, volts, etc., as appropriate, depending on whether the system provides stimulation from current sources or voltage sources. With some SCS systems, the distribution of the current/voltage across the electrodes (including the case of the pulse generator or receiver, which may act as an electrode) may be varied such that the current is supplied via numerous different electrode configurations. In different configurations, different combinations of electrodes may provide current (or voltage) in different relative percentages of positive and negative current (or voltage). Moreover, there may be some electrodes that remain inactive for certain electrode configurations, meaning that no current is applied through the inactive electrode.

Previous SCS technology identified these parameters and effectuated stimulation through an electrode array using specific electrode configurations. These parameters, including distribution (if available), configuration, strength, pulse width, etc., are programmed after implantation in a “fitting” procedure. During the fitting, an extremely large number of possible combinations of stimulation sets may be tested.

In order to test the effectiveness on a particular patient of various stimulation parameters and electrode configurations, it is necessary to provide a series of stimulation parameters in a systematic method. Several such systems exist including the systems disclosed in U.S. Pat. No. 6,393,325, herein incorporated by reference in its entirety, wherein a patient may direct the movement of the stimulus current through a suitable interface.

During a programming session, a clinician and a patient may use a display screen, such as the one described in U.S. Pat. No. 6,622,048, herein incorporated by reference in its entirety. Such a display screen may include a depiction of the human figure divided into body regions. Such body regions may be termed “dermatones,” “body subdivisions,” or “body areas,” or similar language. In use, a pain or paresthesia area or region may be activated by toggling a color box, e.g., red or blue, that is superimposed over the affected body area. One color, e.g., red is used to represent pain; while the other color, e.g, blue, is used to represent paresthesia.

Other display graphics may be developed to make programming efficient, easy, and accurate and to readily convey the values of stimulation parameters to a clinician and patient. Additionally, electrode configuration graphics that are associated with a human figure depiction provide a user with valuable information about a stimulation therapy. Such display graphics are described herein, such as electrode configuration depictions used in connection with a human figure depiction. There is also a need to develop a database that associates specific stimulation sets with pain areas of the body. Such an association would allow a stimulation system to provide specific stimulation sets once a user identifies a region of pain.

### SUMMARY OF THE INVENTION

Embodiments of the present invention describe a tissue stimulation system and devices and methods for programming the stimulation system. The stimulation system may have an implant device comprising an implantable pulse generator having an implantable electrode array connected thereto, the implantable pulse generator having electrical circuitry therein that generates electrical stimulation pulses. This invention is also applicable to a system having an external transmitter that transmits the energy for pulses to an implanted receiver that receives the energy for the pulses and send the pulses to the electrodes implanted adjacent the tissue to be stimulated.

A graphical display for use in a stimulation system may include: (1) a human figure depiction, wherein the human figure is divided into body regions, wherein at least one body region is highlighted with a first color indicating a region of pain and wherein at least one body region is highlighted with a second color indicating a region of paresthesia; and (2) a graphical depiction of the electrical stimulation being generated that causes the paresthesia indicated in the human figure depiction, wherein the graphical depiction may include a depiction of the electrode array, wherein each electrode is represented by an icon depicting whether the corresponding electrode is an anode, a cathode, or turned off.

The depiction of the electrode array may be a columnar representation of the electrode array. Each electrode icon may be a plus sign, a minus sign, or no sign, wherein the plus sign represents an anode, wherein the minus sign represents a cathode, and wherein no sign indicates that the electrode is turned off. The graphical depiction of the electrical stimulation may include at least one plus sign and at least one minus sign. The graphical display may also comprise a numerical value of at least one selected from the group consisting of pulse amplitude, pulse width, and pulse frequency of the pulse being generated that causes the paresthesia indicated. The graphical depiction of the electrode configuration may also comprise a numerical value associated with the sign, wherein the numerical value ranges from 0 to 100, or −100 to +100, wherein the numerical value represents a percentage of current distribution.

Another embodiment of the invention is a method of programming an implant device, the method comprising: (1) creating at least one pain map representing pain sensed by a patient in at least one part of the patient's body; (2) creating at least one paresthesia map representing paresthesia sensed by the patient in response to a particular stimulation set; (3) determining a degree of matching between one of the paresthesia maps and one of the pain maps; (4) if the degree of matching exceeds an acceptable level, associating the stimulation set with the pain map; and (5) retrievably storing the association of the pain map and the stimulation set, wherein subsequent creation or retrieval of the pain map results in stimulation according to the associated stimulation set.

The programming method may thus be used to generate a database, wherein stimulation sets are associated with pain maps. The stimulation sets may include both electrode configuration information and one or more of amplitude, pulse width and pulse rate. The pain and/or paresthesia maps may be created by selecting regions on a displayed human figure by manually moving a computer-generated marker over the part of the body to be selected.

In addition to the implantable pulse generator and the electrode array, a tissue stimulation system may include: (1) means for identifying pain sensed by a patient in at least one specified area of the patient's body; (2) means for identifying paresthesia sensed by the patient in at least one area of the patient's body in response to a particular stimulation set; and (3) a database that includes an association of the stimulation set with the specified area of pain, wherein identification of the specified pain area results in stimulation according to the particular stimulation set.

The means for identifying pain and paresthesia may be an interactive user interface including a depiction of the patient's body as either a grid map or a dermatome map. The stimulation system may include a graphical depiction of the stimulation set, wherein the graphical depiction includes a depiction of the implanted electrode array(s) in a columnar arrangement and wherein each electrode is represented by an icon, wherein each icon comprises a plus sign, a minus sign, or no sign, wherein the plus sign represents an anode, wherein the minus sign represents a cathode, and wherein no sign indicates that the electrode is turned off.

### BRIEF DESCRIPTION OF THE DRAWINGS

The above and other aspects of the present invention will be more apparent from the following more particular description thereof, presented in conjunction with the following drawings wherein:

FIG. 1 depicts a portion of a display screen comprising a human figure depiction and an electrode configuration depiction.

FIG. 2A depicts a portion of a display screen comprising a front view human figure depiction, wherein the areas of pain and paresthesia are indicated.

FIG. 2B depicts a portion of a display screen comprising a back view human figure depiction, wherein the areas of pain and paresthesia are indicated.

FIG. 3 depicts a portion of a display screen representing an electrode as a cathode with 100% current distribution.

FIG. 4 depicts a portion of a display screen representing an electrode as an anode with 60% current distribution.

### DETAILED DESCRIPTION OF THE INVENTION

It is to be understood that this invention is not limited to the particular devices, compositions, methodologies or protocols described, as these may vary. It is also to be understood that the terminology used in the description is for the purpose of describing the particular versions or embodiments only, and is not intended to limit the scope of the present invention which will be limited only by the appended claims.

It must also be noted that as used herein and in the appended claims, the singular forms “a”, “an”, and “the” include plural reference unless the context clearly dictates otherwise. Thus, for example, reference to an “electrode” is a reference to one or more electrodes and equivalents thereof known to those skilled in the art, and so forth. Unless defined otherwise, all technical and scientific terms used herein have the same meanings as commonly understood by one of ordinary skill in the art. Although any methods, devices, and materials similar or equivalent to those described herein can be used in the practice or testing of embodiments of the present invention, the preferred methods, devices, and materials are now described. All publications mentioned herein are incorporated by reference. Nothing herein is to be construed as an admission that the invention is not entitled to antedate such disclosure by virtue of prior invention.

Graphical displays are herein described for use in a stimulation system. These displays may be included in any suitable interface, such as a display screen, a handheld device, a monitor, a laptop, a PDA, or any such device. The displays may be interactive, such as a touch screen, or may be simply informative. The systems incorporating the displays may employ a mouse, joystick, or stylus for input. Both a human figure and an electrode configuration may be depicted in the present embodiments. The graphical displays may be used as part of a stimulation system or related devices to convey stimulation parameters to a user, such as during the fitting or programming of the stimulation system. Additionally, the graphical displays aid the users in identifying pulses that effectively treat the pain area.

The various components of an exemplary SCS system may include an implantable pulse generator (IPG) (or an external transmitter and internal receiver) and hand-held programmer (HHP) used with such system. Implantable components may include an implantable pulse generator (or receiver), one or more electrode arrays, and (as needed) one or more extensions to connect the array(s) to the IPG. Such implantable components, external devices and circuitry are more fully described in U.S. Pat. No. 6,622,048.

A programming system may include, as described in U.S. Pat. No. 6,622,048, a clinician programmer coupled to a directional device. The clinician programmer typically interfaces with a patient hand-held programmer (HHP) in communicating with the implanted pulse generator. However, other types of communication links between the clinician programmer (i.e., the programming computer) and the IPG may be utilized.

The programming system maintains a patient data base, and is able to program all features of the implant device in a simple and intuitive manner. Preprogrammed into the data base, along with information about the patient, is known information regarding anatomical relationships between the spine and the body (such as dermatomes). Additionally, the system allows threshold measurements to be made, operational electrodes to be identified, and is able to interface directly with the patient.

The programming system may use a joystick accessory, or equivalent directional device, which allows the patient to interface with a computer (e.g., programmed to function as the clinician programmer), or other processor (e.g., a hand-held computer, such as a PalmPilot® computer, or equivalent) so as to allow the patient, or other medical personnel assisting the patient, to configure electrodes and adjust various stimulation parameters and to identify regions of the body where pain is present and where paresthesia is felt. One suitable directional programming device is described in more detail in U.S. Pat. No. 6,052,624, entitled “Directional Programming for Implantable Electrode Arrays”, which is incorporated herein by reference.

The directional programming device may take many forms. For purposes of the present invention, any device that allows a computer-generated cursor (or other indicator) to move about on the display screen of the computer as controlled by the user will suffice. Representative directional programming devices include keys on a keyboard (e.g., arrow keys), a joystick, a mouse, a track ball, a touch-sensitive screen over which the user's finger may be moved, voice commands in combination with voice recognition software, light sensors on which a light beam, e.g., a laser wand, may be directed, and the like.

Additional details associated with the Clinician's programming system and the patient handheld programmer (HHP) may be found in the previously referenced U.S. Pat. No. 6,622,048.

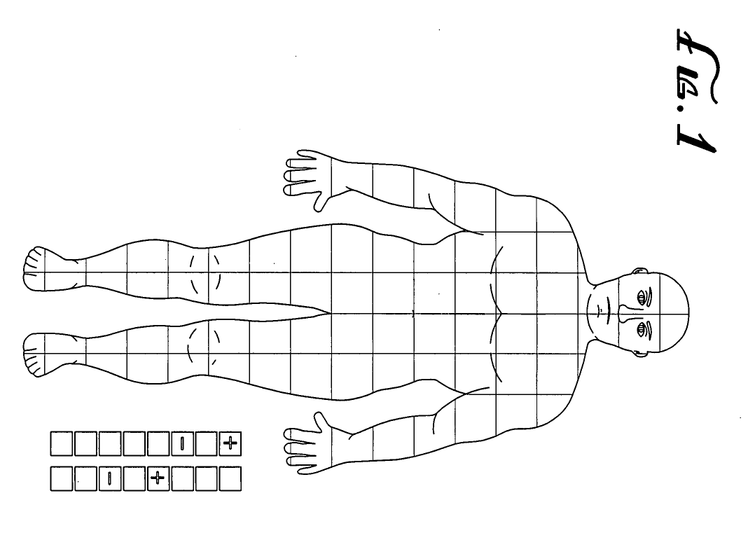

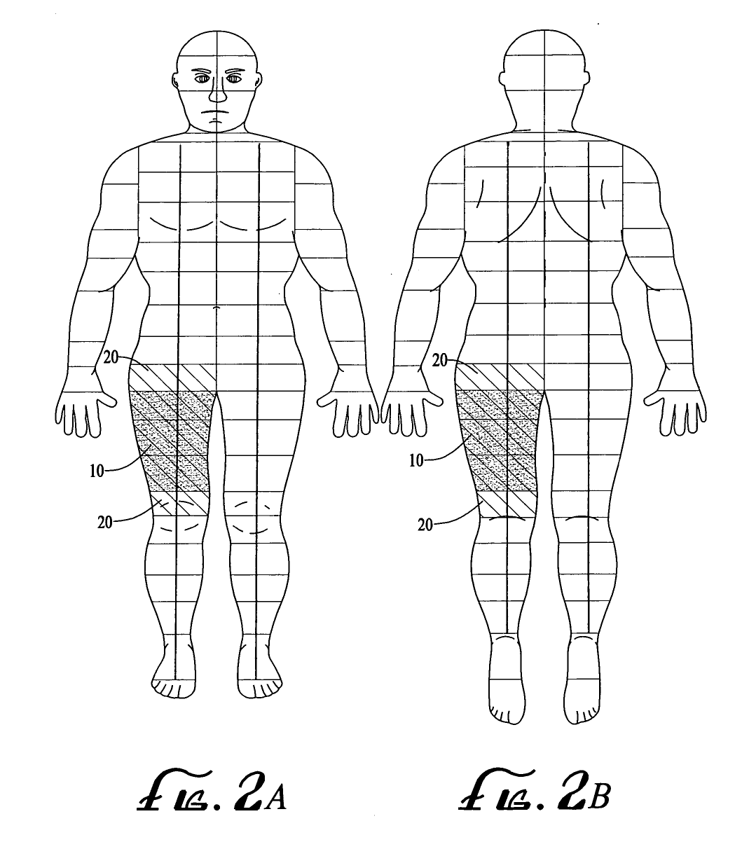

Pain and/or paresthesia mapping is available to identify effective electrode configurations for treating a targeted pain area. To aid in this process, a human figure is displayed on the display screen associated with the programming computer. Refer to FIGS. 1, 2A and 2B. This human figure is divided into body regions. Such body regions may also be known as “body subdivisions,” “body areas,” or similar language, which regions may be based on dermatomes. Alternatively, the human figure may be divided into a grid map of squares, such as seen in FIG. 1. While the human figure shown in FIG. 2 is shown as being divided into over sixty (60) such regions, such number of regions is only exemplary.

A pain or paresthesia area or region may be activated by toggling a color area, e.g., red or blue, that is superimposed over the affected body area. One color, e.g., red, may be used to represent pain; while another color, e.g, blue, may be used to represent paresthesia. As a mouse button is clicked or a button or key is pressed while the cursor is moved over different body segments or regions, such segments change color. The paresthesia color may be translucent (top layered) so that pain segments can be seen through it. FIG. 2A depicts a frontal view of the human figure, while FIG. 2B depicts a back view. As seen in FIGS. 2A and 2B, the pain color is depicted as darker [10] and may be seen through the paresthesia color [20], which may be translucent.

Body segments may be selected individually, or as a group at intersections. In some embodiments, an “active” color may be set, so that when clicking on a segment, the active color is toggled off and on without affecting an alternate color. The object is to match or map the paresthesia segments with the pain segments. Such pain/paresthesia mapping feature may advantageously be used with expert algorithms to automate the programming process. Alternatively, the patient and clinician/physician may simply work together and use a trial-and-error procedure in order to best fit the paresthesia segments with the pain segments.

To perform the pain/paresthesia mapping in accordance with the invention, the patient first identifies a region of pain. Such pain region may be identified by simply moving the cursor over areas and clicking a button, e.g., a mouse button or a keyboard button.

Once the patient has identified a pain region, the programming computer may use known data regarding the relationships between the electrode physical locations and the body to select a first electrode combination through which a stimulus of selected operating parameters may be applied. As explained more fully herein, this database may include empirically tested electrode configurations that are associated with the area of pain.

Once selected, a stimulus having the selected operating parameters is applied to the selected electrode combinations to test whether the resulting paresthesia is in the same location as the pain region. Thus, for example, after feeling or sensing the paresthesia, the patient identifies the location of the paresthesia on the human figure, as described earlier.

Once the patient has identified the paresthesia region, the programming computer uses this information, in combination with other information stored therein, to determine what modifications need to be made to the stimulation parameters or electrode selection in order to steer the paresthesia region over the pain region. Alternatively, the user (e.g., patient or clinician) uses a program and directional device to “navigate” along the electrode array to find an overlapping electrode configuration. Alternatively or additionally, the programmer's electrode and parameter selection tools allow the user to vary the stimulation parameters to better address the pain area. The newly selected stimulation set, including the selected electrode configuration, may produce a new paresthesia region that matches with the pain region.

Once an acceptable match has been obtained between the pain region and the paresthesia region (e.g., when the patient's pain has been replaced or mostly replaced by paresthesia), the programming data, e.g., the stimulation set, including the selected electrode configuration and stimulation parameters, may be programmed into the memory of the implantable pulse generator or other internal or external device so that such data can thereafter be used to control the stimulation delivered by the system, and to further aid in subsequent programming sessions.

One programming approach is detailed more fully in U.S. Pat. No. 6,622,048, herein incorporated by reference. In this approach, the programming involves creating a database that maps various electrode configurations to paresthesia regions of the body. Using a display of a human figure on a screen of the programming computer, the user selects at least one region on the displayed human figure where the patient feels pain. From the database, the stimulation system selects stimulation parameters to produce paresthesia in the region of pain. The pulse generator generates the stimulation pulses and delivers the pulses to the chosen combination of electrodes. The user identifies at least one region of paresthesia on the displayed human figure where the stimulation pulses produce paresthesia. The stimulation system determines the degree of mismatch between the region of paresthesia and the region of pain. If the degree of mismatch exceeds an acceptable level, the stimulation system selects a new combination of electrodes and stimulation parameters based on the level of mismatch. This mapping procedure continues until the degree of mismatch between the region of paresthesia and the region of pain is reaches zero or some acceptable level.

A database may be created that associates a pain map with a stimulation set. Such a database may be created by the repetition of the following steps: (1) creating at least one pain map representing pain sensed by a patient in at least one part of the patient's body, (2) creating at least one paresthesia map representing paresthesia sensed by the patient in response to a particular stimulation set, (3) determining a degree of matching between one of the paresthesia maps and one of the pain maps, (4) if the degree of matching meets an acceptable level, associating the stimulation set with the pain map, and (5) retrievably storing the association of the pain map and the stimulation set, wherein subsequent creation or retrieval of the pain map results in stimulation according to the stimulation set. Through these steps, a database may be created that associates stimulation sets with pain maps. Such a database may be specific for each patient or may be shared across patients. The stimulation sets may include both the electrode configuration and parameters such as pulse width, pulse rate, and amplitude.

This database may be created in a preliminary stimulation (programming or fitting) session. At subsequent stimulation sessions, this database may be used to deliver effective stimulation sets to a patient that has identified a region of pain. Other programming algorithms may be employed with the graphical displays of the present invention. Additionally, other mechanisms of pain mapping are also suitable in the systems of the present invention.

The programming method may thus be used to generate a database, wherein stimulation sets are associated with pain maps. The stimulation sets may include both electrode configuration information and one or more of amplitude, pulse width and pulse rate. The pain and/or paresthesia maps may be created by selecting regions on a displayed human figure by manually moving a computer-generated marker over the part of the body to be selected, and selecting appropriate regions.

For example, if the degree of matching between the paresthesia region and the pain region is greater than an acceptable threshold, e.g., if 90% of the pain region overlaps with the paresthesia region, then a match condition is assumed. In such an instance, the electrode selection and stimulation parameters that resulted in such match condition may be sent to and stored in the IPG, or other implanted or external device, to control the operation of the device in a manner that regularly overlays the paresthesia region on the pain region. With the programming of the implant device, any database in the computer memory is preferably updated with the data that produced the match condition. If the degree of matching is below an acceptable threshold, e.g., less than 90% matching, than the computer selects another parameter set(s) and applies pulses to the patient until such levels of matching are achieved. Such reselection is described more fully in U.S. Pat. No. 6,622,048.

The database that associates stimulation sets with areas of pain may thus be incorporated into any suitable stimulation system. For example, in addition to the stimulation pulse generator and the electrode array, the tissue stimulation system may include: (1) means for identifying pain sensed by a patient in at least one area of the patient's body; (2) means for identifying paresthesia sensed by the patient in at least one area of the patient's body in response to a particular stimulation set, wherein the area of paresthesia substantially matches the area of pain; and (3) a database that includes an association of the stimulation set with the area of pain, wherein identification of the pain area results in stimulation according to the stimulation set.

Thus, the creation of the database that associates the pain map to specific stimulation sets and the use of this database involve the use of a displayed human figure. The human figure depiction thus includes a human figure divided into a plurality of body regions, wherein at least one body region is shown with a first color indicating a region of pain and wherein at least one body region is shown with a second color indicating a region of paresthesia. The second color may be translucent so the first color is visible “below” the second color, or such that a region that was selected as a region of pain and also as a region of paresthesia is shown with a third color. In addition to the displayed human figure, a graphical display of the electrode combination that is supplying the perceived paresthesia may be included on a display screen.

Previous graphical displays included a simplified electrode array on the screen. For example, in U.S. Pat. No. 6,622,048, the array is displayed on the screen with point and click selectable electrodes. For example, clicking on an electrode may be used to specify a cathode, anode, or a neutral (floating or non-connected) electrode, by cycling through the choices as the electrode is clicked. Cathode, anode and neutral selections are indicated by a color change, plus/minus/no sign, or the like.

A visual representation of the electrode configuration being applied to a patient that causes the perceived paresthesia may aid in the effective selection of pain therapies. The patient may be allowed to see which electrodes of the array are supplying the pulse. The graphical depiction of the electrode configuration thus describes, in part, the pulse being generated that causes the paresthesia indicated in the human figure depiction.

Electrode configurations that are causing the paresthesia may be depicted and associated with the pain mapping procedure. As used herein, an “electrode configuration” refers to a polarity and/or to a relative distribution of current or voltage applied through the electrodes of the electrode array. Electrodes may be positive, negative, or turned off, such that a subset of anodes and cathodes are created within the electrode array. A polarity of each electrode may be a positive or negative “1” or a fraction thereof. For example, one electrode of the electrode array may have a polarity of negative “1” (cathode), while another electrode may have a polarity of positive “1” (anode).

Alternatively, a polarity may be spread out among different electrodes, for example, such that one electrode has a polarity of +0.75, while the other electrode(s) have +0.25. This distribution is known as polarity “distribution” or “percentage” among the electrodes of an electrode array. In the above examples, if an electrode has a polarity of negative 1, it is a cathode with 100% of the negative polarity distribution. If an electrode has a polarity of +0.75, it is an anode with 75% of the polarity distribution (with one or more additional electrodes accounting for the remaining 25% of the positive polarity distribution). Thus, a numerical value may be easily associated with a polarity distribution. In the case of current-controlled electrodes, in this example 75% of the anodic current would emanate from the first anode and 25% of the anodic current from the remaining anode(s). In the case of voltage-controlled electrodes, in this example the voltage magnitude of the first anode (e.g. +3.0 volts or 75%) would be three times that of the other anode(s) (+1.0 volts or 25%).

The total current applied through each electrode may be about 1 to about 13 milliamps, up to a “grand total” of 20 milliamps applied through all active electrodes combined. The values of the electrode configuration therefore represent a percentage of this grand total current applied through an individual electrode. Alternatively, the stimulation may be measured by voltage applied to the electrodes, or by a combination of voltage and current to different electrodes.

During a stimulation session, it may be helpful for a user to know whether a particular electrode of the electrode array is operating as an anode, a cathode, or is turned OFF. It may also be important for the user to know what percentage of the current (or voltage) is being applied through a particular electrode, when applicable. Such knowledge allows a user to evaluate and/or to change the electrode configurations being applied in order to meet therapeutic objectives.

A representative programming window may include the electrode arrays depicted in a two column arrangement as seen in FIG. 1. Each electrode is represented by an icon having a rectangular or square shape in one of two columns of the 16 icons of the two electrode arrays, each array having eight electrodes. Each icon includes a sign, representing an anode or cathode, or by a void or blank, representing that the electrode is OFF. The sign is selected from the group consisting of a plus sign and a minus sign, wherein the plus sign represents an anode and wherein the minus sign represents a cathode. Other options for depicting cathodes, anodes, and electrodes that are off include different colors, shapes, symbols, words, and the like. Additionally, a numerical value may be associated with the icon, wherein the numerical value or equivalent represents a percentage of current distribution. The numerical value may range from 0 to 100, or from −100 to +100, thus representing the percentage of current being supplied to that particular electrode. Equivalents may include charts, such as pie charts, or graphs, such as bar graphs, depicting percentages.

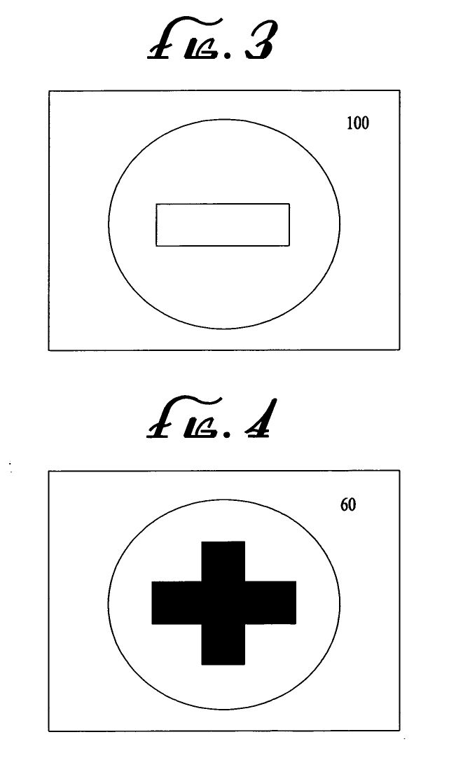

FIG. 3 depicts a portion of an example display screen representing an electrode as a cathode with 100% of the cathodic current distribution. This design is generally a square/rectangular shaped icon including a sign and a numerical value. This icon informs a user that this particular electrode is acting as a cathode and that this electrode is the only cathode in the array at this time. This information is readily interpreted by the user through the use of the minus sign and the numerical value of 100. As another example, FIG. 4 represents an electrode as an anode having 60% current distribution. This means that another electrode or electrodes in the array must account for the other 40% of the anodic current being applied.

Each electrode of an electrode array may have a design featuring the sign and the numerical value. These graphics may be organized on a user interface in any suitable manner. For example, the electrode depictions may be arranged in sequential columnar order, such as the electrode arrays depicted in FIG. 1.

Furthermore, the display may also include the use of one or more colors to represent one or more characteristics of the particular electrode. For example, polarity may be shown with colors instead of in addition to positive and negative signs. As another example, various colors may be used to indicate that a particular electrode has a high or low impedance value. Impedance (defined as voltage divided by current) may be used as a measure of system stability and hardware connectivity. For a spinal cord implantation, the electrode impedance will typically range between about 400 ohms and 1000 ohms. Implanted electrical stimulation systems (including leads and electrodes) convey electrical pulses of known energy to the target tissue to be excited. The target tissue represents a known electrical load into which the electrical energy associated with the stimulation pulse is to be delivered. If the impedance is too high, a connector and/or lead that connects with the electrode may be open or broken. If the impedance is too low, there may be a short circuit somewhere in the connector/lead system. In either event (too high or too low impedance), the system may be unable to perform its intended function. Impedance measurement and its importance in stimulation systems are more thoroughly detailed in U.S. Pat. No. 6,516,227, herein incorporated by reference in its entirety.

As another example, other display indicators may be used to show polarity and/or polarity percentages, and/or impedance, such as various colors, charts, graphs, shapes, patterns, symbols, words or the like. For example, a pie chart or bar chart may be used to show the percentage of the polarity of each electrode. As another example, an electrode having a high impedance value may have an overlaying pattern associated with its icon such as a line grid. As another example, an icon may include the letter “i” to indicate that the electrode has a high impedance value. Other examples may be readily ascertained by one skilled in the art.

Thus, for example, the color red may be used to indicate that an electrode has a high impedance value. A lighter shade of red or a different color may be used to represent a low impedance. The numerical value of the impedance may also be shown. If an electrode has too high or low an impedance value, a fitting program may automatically block the supply of current (or voltage) to this electrode. The high or low impedance indicator alerts the user to avoid the particular electrode(s) or that the system is avoiding the electrode(s).

Other display icons and features may be incorporated into the display screen in stimulation sessions. For example, a numerical value for the pulse amplitude, pulse width, and pulse frequency may be included in the display. Means for interactively adjusting these parameters may also be included, such as “up” and “down” arrows, sliding scales, and dials.

While the invention herein disclosed has been described by means of specific embodiments and applications thereof, numerous modifications and variations could be made thereto by those skilled in the art without departing from the scope of the invention set forth in the claims. For example, the methods discussed above are not limited to spinal cord stimulation systems and may be used with many kinds of stimulation systems such as cochlear implants, cardiac stimulation systems, peripheral nerve stimulation systems, muscle tissue stimulation systems, brain stimulation systems and microstimulators.

1. A tissue stimulation system having an implant device capable of delivering electrical stimulation pulses through one or more electrodes, the system comprising:
a display screen for use in a stimulation session, wherein the display screen comprises:
a human figure depiction, wherein the depiction is divided into body regions, wherein at least one body region is shown in a first color indicating a region of pain and wherein at least one body region is shown in a second color indicating a region of paresthesia; and
a graphical depiction of the electrical stimulation pulse being generated that causes the paresthesia indicated in the human figure depiction, wherein the graphical depiction includes a depiction of the one or more electrodes, wherein each electrode is represented by an icon depicting whether the corresponding electrode is an anode, a cathode, or turned off.

2. The system of claim 1, wherein the depiction of the electrodes is a columnar arrangement.

3. The system of claim 1, wherein each electrode icon includes a plus sign, a minus sign, or no sign, wherein the plus sign represents an anode, wherein the minus sign represents a cathode, and wherein no sign indicates that the electrode is turned off.

4. The system of claim 1, wherein the tissue stimulation system is a spinal cord stimulation system.

5. The system of claim 1, wherein the graphical depiction of the electrical stimulation further comprises a numerical value of at least one parameter selected from the group consisting of pulse amplitude, pulse width, and pulse frequency of the electrical stimulation pulses being generated that cause the paresthesia indicated.

6. The system of claim 1, wherein each icon includes a representation of polarity percentage, wherein the representation comprises at least one of a numerical value, a chart and a graph.

7. The system of claim 6, wherein the numerical value is between 0 and 100.

8. The system of claim 1, wherein an electrode with a high impedance state is so indicated by at least one of a color, a shape, a chart, a graph, a pattern, one or more words, and one or more symbols.

9. The system of claim 8, wherein the color is red.

10. The system of claim 1, wherein the first color is red.

11. The system of claim 1, wherein the second color is blue.

12. The system of claim 1, wherein the region of pain is substantially similar to the region of paresthesia.

13. The system of claim 1, wherein the second color is translucent so that the presence of the first color may be seen therethrough.

14. A method of programming a tissue stimulation apparatus having at least one electrode implanted in a patient, wherein the apparatus delivers stimulation pulses to the patient's tissue through the at least one electrode, wherein the programming method comprises:
creating at least one pain map representing pain sensed by a patient in at least one part of the patient's body;
creating at least one paresthesia map representing paresthesia sensed by the patient in response to a particular stimulation set;
determining a degree of matching between one of the paresthesia maps and one of the pain maps;
if the degree of matching exceeds an acceptable level, associating the stimulation set with the pain map; and
retrievably storing the association of the pain map and the stimulation set, wherein subsequent creation or retrieval of the pain map results in stimulation according to the associated stimulation set.

15. The method of claim 14, wherein the creating steps are performed by selecting regions on a displayed human figure, including manually moving a computer-generated marker over the part of the body to be selected.

16. A tissue stimulation system having an implant device capable of delivering electrical stimulation pulses through one or more electrodes, the system comprising:
means for identifying pain sensed by a patient in at least one specified area of the patient's body;
means for identifying paresthesia sensed by the patient in at least one area of the patient's body in response to a particular stimulation set; and
a database that includes an association of the particular stimulation set with the specified area of pain, wherein identification of the specified pain area results in stimulation according to the particular stimulation set.

17. The system of claim 16, wherein the means for identifying pain and paresthesia is an interactive user interface including a depiction of the patient's body.

18. The system of claim 17, wherein the depiction is a grid map of the patient's body.

19. The system of claim 17, wherein the depiction is a dermatome map of the patient's body.

20. The system of claim 17, further comprising a graphical depiction of the electrical stimulation being generated that causes the paresthesia indicated in the depiction of the patient's body, wherein the graphical depiction includes a depiction of the electrodes, wherein each electrode is represented by an icon depicting whether the corresponding electrode is an anode, a cathode, or turned off.
